(19) United States
(12) Patent Application Publication
(10) Pub. No.: US 2025/0387224 A1
(43) Pub. Date: Dec. 25, 2025

(54) POLYMER VALVE LEAFLET MATERIAL, VALVE LEAFLET, VALVE AND PREPARATION METHOD THEREFOR

(71) Applicant: VENUS MEDTECH (HANGZHOU) INC., Hangzhou (CN)

(72) Inventors: Lichao Wang, Hangzhou (CN); Xing Wei, Hangzhou (CN); Manman Zhao, Hangzhou (CN); Dajun Kuang, Hangzhou (CN); Yuting Zhu, Hangzhou (CN)

(21) Appl. No.: 19/254,014

(22) Filed: Jun. 30, 2025

Related U.S. Application Data
(63) Continuation of application No. PCT/CN2023/112855, filed on Aug. 14, 2023.

(30) Foreign Application Priority Data
Dec. 30, 2022 (CN) 202211721483.5

(51) Int. Cl. A61F 2/24; A61L 27/36; B29C 49/04

(52) U.S. Cl. CPC A61F 2/24; A61L 27/36; B29C 49/04

(57) ABSTRACT

Disclosed are a polymer valve leaflet material, a valve leaflet, a valve and a preparation method thereof. The polymer valve leaflet material is made of polyurethane and has a tensile strength in a range of 35 MPa to 60 MPa, an elastic modulus in a range of 15 MPa to 40 MPa, a softness in a range of 20° to 50°, and a thickness in a range of 0.10 mm to 0.20 mm.

### CROSS-REFERENCE TO RELATED APPLICATIONS

The present disclosure is a Continuation Application of PCT Application No. PCT/CN2023/112855, filed on Aug. 14, 2023, which claims priority to Chinese Patent Application No. 202211721483.5, filed on Dec. 30, 2022, the entire contents of which are hereby incorporated by reference.

### TECHNICAL FIELD

The present disclosure relates to the technical field of medical materials, and in particular to a polymer valve leaflet material, a valve leaflet, a valve, and a preparation method thereof.

### BACKGROUND

In the art, the application of polymer materials in valves is becoming increasingly widespread. However, it is generally challenging for the polymer materials used in valves to simultaneously meet the requirements in terms of mechanical properties, biostability and biocompatibility. Typically, polymer materials with excellent mechanical properties often fail to meet the requirements for biostability and biocompatibility simultaneously, whereas those with excellent biostability and biocompatibility struggle to meet the requirements for mechanical properties. Moreover, even under minimal stress, polymer materials are prone to relative slippage (known as creep) between molecular chain segments over time. This causes the elongation of the valve leaflets, which results in incomplete closure and significant regurgitation.

Polyurethane has been used as the valve leaflet material for a long time. Existing technologies typically seek a balance or trade-off between biocompatibility, biostability, and mechanical performance. Currently, rapidly advancing research on the implantable polyurethane valve material mainly focuses on the following two types.

The first is PDMS-PU, in which polydimethylsiloxane (PDMS) is added to polyurethane to improve the biostability and biocompatibility of polyurethane. However, this comes at the cost of some mechanical properties. By adjusting the ratio of raw materials, a basic balance between softness and strength is achieved. At present, the polyurethane material with good performance has a tensile strength in a range of 35 MPa and an elastic modulus in a range of 18 MPa.

The second is POSS-PCU, in which the cage-shaped POSS is added to polyurethane to improve the biostability and biocompatibility of polyurethane. Silicon atoms on the nanoparticles tend to accumulate on the surface of the material, which significantly improves the biocompatibility. Additionally, the cage-shaped POSS plays a role similar to filler, somewhat increasing the strength of the polyurethane. However, this also leads to a significant rise in elastic modulus and a marked increase in hardness.

The polymer materials used for valves must be soft enough to meet the requirements of valve leaflet fluid mechanics, yet possess sufficient strength and a sufficiently high elastic modulus to improve fatigue resistance and creep resistance for long-term durability. The existing planar cutting process for biofilm imposes stringent requirements on the material in terms of strength and softness. However, due to the inherent limitations of these materials in meeting the required performance, valves made from biofilm cannot function properly. As a result, polyurethane materials are reinforced with fabrics to meet mechanical performance requirements.

### Technical Problems

Fabric reinforcement primarily relies on fabric to bear mechanical stress, allowing the polyurethane valve leaflet which inherently lacks intrinsic strength to meet mechanical requirements. Meanwhile, the fabric helps restrain polyurethane creep. However, as the valve undergoes repeated opening and closing, the surface polyurethane will wear out over a long period. Once it is peeled off, the fabric may be exposed, which leads to problems such as thrombus calcification. Meanwhile, the point where the valve leaflet is subjected to the greatest force is the center of the free edge. Due to interstitial gaps in the fabric, the outermost periphery of the free edge is coated with polyurethane, which struggles to withstand the shear stress at the center of the free edge. This results in notches on the free edge and increases the risk of polyurethane peeling.

### SUMMARY

Based on this, the present disclosure provides a polymer valve leaflet material that possesses the biocompatibility, biostability and mechanical properties that meet requirements for the valve leaflet.

Provided is a polymer valve leaflet material, wherein the polymer valve leaflet material is made of polyurethane. The polymer valve leaflet material has a tensile strength in a range of 35 MPa to 60 MPa, an elastic modulus in a range of 15 MPa to 40 MPa, a softness in a range of 20° to 50°, and a thickness in a range of 0.10 mm to 0.20 mm.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, the permanent deformation of the polymer valve leaflet material ranges from 5% to 10%.

The present disclosure further provides a method for preparing a polymer valve leaflet material, including the following steps:
applying force to the polymer membrane to stretch the polymer membrane in a first direction to a target dimension of 120% to 300% of its original dimension, restraining the deformation of the polymer membrane in a second direction, wherein the second direction is perpendicular to the first direction, and continuously applying the force for at least 30 minutes after the polymer membrane reaches the target dimension.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, the polymer membrane is elongated at a rate in a range of 100 mm/min to 500 mm/min.

Optionally, the polymer membrane is elongated at a rate in a range of 100 mm/min to 200 mm/min.

Optionally, the target dimension of the polymer membrane is 150% to 200% of the original dimension.

Optionally, after the polymer membrane reaches the target dimension, the force is continuously applied for 30 minutes to 180 minutes.

Optionally, the polymer membrane before being elongated has a thickness in a range of 0.15 mm to 0.40 mm.

Optionally, the polymer membrane before being elongated has a thickness in a range of 0.15 mm to 0.30 mm.

Optionally, the polymer membrane before being elongated has a tensile strength in a range of 20 MPa to 35 MPa, an elastic modulus in a range of 10 MPa to 30 MPa, a softness in a range of 10° to 20°, and a permanent deformation in a range of 10% to 40%.

Optionally, the polymer membrane before being elongated is prepared from polyurethane by casting molding.

Optionally, the polymer membrane is in a planar or curved shape during the stretching process.

Optionally, during the stretching process, two opposite side edges of the polymer membrane are connected to each other to form a circumferentially closed tubular structure.

Optionally, the two opposite side edges are integrally connected or indirectly connected via a connecting member.

Optionally, when the edges are indirectly connected, the polymer membrane spans at least half of the circumference of the tubular structure, with the remaining portion of the circumference being constituted by the connecting member.

Optionally, the polyurethane molecular chains include hard and soft segments, wherein the soft segment content is in a range of 40% to 70%, and the rest is the hard segment. The soft segment is at least one selected from the following: polyether diol, polycarbonate diol, polyester diol, and polysiloxane diol, and the hard segment is derived from isocyanate with an R value in a range of 1.0 to 1.1.

Optionally, the isocyanate is at least one selected from the following: TDI, HDI, MDI, NDI, PPDI, IPDI, and XDI.

Optionally, the polyurethane molecular chain further includes a chain extender, and the chain extender is at least one selected from the following: ethylene glycol, butanediol, hexanediol, octanediol, and ethylenediamine.

Optionally, the polymer membrane is planar and prepared by casting in a mold, wherein the mold has a flat bottom surface and side walls standing on the bottom surface and defining the boundaries of the polymer membrane.

Optionally, a polyurethane solution with a concentration in a range of 3 wt. % to 40 wt. % is poured into the mold, and the solvent is evaporated to obtain the polymer membrane.

Optionally, the concentration of the polyurethane solution is in a range of 5 wt. % to 30 wt. %.

Optionally, the solvent of the polyurethane solution is volatilized at 30° C. to 100° C. in a nitrogen atmosphere.

Optionally, the solvent of the polyurethane solution is at least one selected from the following: DMAc, DMF, DMSO, THF and toluene.

Optionally, the polymer membrane is a tubular polymer membrane, and the method for preparing the tubular polymer membrane includes:

Step 1, applying a polyurethane solution onto the surface of the tubular mold and volatilizing the solvent to obtain a polyurethane membrane; and

Step 2, repeating step 1 three to six times to obtain a tubular polymer membrane with a predetermined thickness on the surface of the tubular mold.

Optionally, in step 1, the polyurethane solution is applied to the surface of the tubular mold by at least one of coating and dipping.

Optionally, in step 1, the tubular mold is rotated continuously at a speed in a range of 1 r/min to 30 r/min while being soaked in the polyurethane solution to achieve applying the polyurethane solution onto the surface of the tubular mold.

Optionally, in step 1, the solvent is volatilized under a nitrogen atmosphere and dried at 30° C. to 100° C.

Optionally, the tubular mold is a rotatable body, with its generatrix being straight or curved.

Optionally, the rotation axis of the tubular mold is arranged horizontally.

Optionally, the diameter of the tubular mold is in a range of 15 mm to 35 mm.

Optionally, the tubular polymer membrane is soaked in water for 1 hour to 12 hours together with the tubular mold, and then the tubular polymer membrane is peeled off from the surface of the tubular mold.

Optionally, the first direction and the second direction are coplanar and perpendicular, or the first and second directions are arranged in three-dimensional space, in which one of the first and second directions corresponds to the axial direction while the other of the first and second directions corresponds to a circumferential direction around that axis.

Optionally, the deformation of the polymer membrane is W1 when it is elongated to the target dimension in the first direction, the deformation of the polymer membrane in the second direction is W2, wherein W2/W1<30%. More preferably, W2/W1<10%. Even more preferably, W2/W1<5%.

Optionally, the deformation of the polymer membrane in the second direction is restrained by applying a restraining force to two opposite sides of the polymer membrane in the second direction.

Optionally, a first device is used to elongate the planar polymer membrane, wherein the first device includes:
a first clamp for fixing the polymer membrane in a second direction; and
a second clamp for applying force to the polymer membrane in a first direction.

Optionally, the first clamp provides multiple force-applying sites for the same-side edge of the polymer membrane, and the distance between the force-applying sites along the first direction is adjustable.

Optionally, the first clamp is a press roller.

Optionally, there are two sets of press rollers, each corresponding to one of the two opposite edges of the polymer membrane, with the axial direction of each press roller parallel to the second direction.

Optionally, each set of press rollers includes at least two press rollers, the contact areas where press rollers interact with the polymer membrane define contact lines, and the distance between the contact lines of two adjacent press rollers is in a range of 50 mm to 200 mm.

Optionally, each press roller includes a fixed shaft and a rotating roller rotatably mounted on the fixed shaft.

Optionally, there are two pairs of second clamps, each corresponding to one of the two opposite edges of the polymer membrane, and the two pairs of second clamps clamp the polymer membrane and move in opposite directions to stretch the polymer membrane in the first direction.

Optionally, each pair of second clamps includes multiple clamping parts arranged at intervals, and the distance between two adjacent clamping parts ranges from 5 mm to 20 mm.

Optionally, two opposite edges of the planar polymer membrane are provided with fabric-reinforced borders, and the fabric-reinforced borders are connected with traction lines for engaging with the second clamp.

Optionally, a second device is used to stretch the tubular polymer membrane, wherein the second device includes:
a radially expandable balloon, having a tubular segment around which the tubular polymer membrane is mounted; and
an infusion device provided for delivering fluid into the balloon.

The first direction may be understood as the circumferential direction of the tubular segment, and the second direction may be understood as the axial direction of the tubular segment.

Optionally, the two axial sides of the tubular polymer membrane may be fixed to the outer periphery of the tubular segment, for example, by means of adhesion.

Optionally, a third device is used to stretch the tubular polymer membrane, and the third device includes:
a support column, configured to mount the tubular polymer membrane thereon;
two clamping members, each clamping one axial end of the polymer membrane to stretch the polymer membrane in the first direction; and
at least one movable ring, slidably mounted around the support column, with at least one clamping member disposed on the movable ring.

The first direction may be understood as the axial direction of the support column, and the second direction may be understood as the axial direction of the support column.

The cross section of the support column is not strictly limited and may adopt a configuration with a smooth outer contour such as a circle or ellipse. The support column may be solid or hollow, as long as it provides sufficient structural strength.

Optionally, two movable rings are mounted around the support column, with each clamping member disposed on the corresponding movable ring.

Optionally, a movable ring is mounted around the support column, with one clamping member fixed to the support column and the other fixed to the movable ring.

Optionally, the support column is provided with a guide device for defining the path of the movable ring as axial along the support column.

Optionally, two axial ends of the tubular polymer membrane are respectively provided with fabric-reinforced borders, and the fabric-reinforced borders are connected with traction lines for cooperating with the clamping member.

Optionally, a fourth device is used to stretch the tubular polymer membrane, and the fourth device includes:
multiple support members movable toward and away from each other, each support member being acting on the inner surface of the tubular polymer membrane; and
a drive mechanism for driving at least two support members to move away from each other.

The present disclosure further provides a method for preparing a polymer valve leaflet, including:
soaking the polymer valve leaflet material in water for at least 4 hours and cutting it to obtain the polymer valve leaflet.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, the polymer valve leaflet material is soaked in water at 30° C. to 40° C.

Optionally, the polymer valve leaflet material is soaked in water at 37° C.

Optionally, the valve leaflet includes: a fixed edge fixedly connected to the stent, and a free edge configured to cooperate with the free edge of the adjacent valve leaflet in controlling the blood flow channel, wherein the polymer membrane is elongated in a first direction, and the first direction is parallel to the extending direction of the free edge of the valve leaflet.

Optionally, the polymer valve leaflet material has a thickness in a range of 0.10 mm to 0.20 mm.

The present disclosure further provides a polymer valve leaflet prepared by the aforementioned preparation method for the polymer valve leaflet.

Optionally, the valve leaflet material has a tensile strength in a range of 35 MPa to 60 MPa, an elastic modulus in a range of 15 MPa to 40 MPa, a softness of in the range 20° to 50°, a permanent deformation in a range of 5% to 10%, and the polymer valve leaflet material has a thickness in a range of 0.10 mm to 0.20 mm.

The present disclosure further provides a polymer valve, including:
a stent, defining a blood flow channel therein; and
one or more valve leaflets, wherein the valve leaflets are made of the polymer valve leaflet material.

Optionally, the valve leaflet material has a tensile strength in a range of 35 MPa to 60 MPa, an elastic modulus in a range of 15 MPa to 40 MPa, a softness in a range of 20 to 50°, a permanent deformation in a range of 5% to 10%, and the polymer valve leaflet material has a thickness in a range of 0.10 mm to 0.20 mm.

### Beneficial Effects

The present disclosure provides a polymer valve leaflet material that simultaneously meets the requirements of biocompatibility, biostability and mechanical properties for the valve leaflet.

### BRIEF DESCRIPTION OF DRAWINGS

FIG. 1 shows a schematic view of a first device stretching a polymer membrane;

FIG. 2 shows a schematic view of a first device stretching a polymer membrane, wherein the polymer membrane has been reinforced;

FIG. 3a shows a schematic view of a second device stretching a polymer membrane in an initial state;

FIG. 3b shows a schematic view of a second device stretching a polymer membrane in a final state;

FIG. 4 shows a schematic view of cutting a tubular polymer membrane to form a valve leaflet;

FIG. 5 shows a schematic view of a third device stretching a polymer membrane;

FIG. 6a shows a schematic view of a fourth device stretching a polymer membrane in an initial state;

FIG. 6b shows a schematic view of the fourth device stretching the polymer membrane in a final state.

In the figures: 110, press roller; 120, second clamp; 200, polymer membrane; 210, fabric-reinforced border; 220, traction line; 300, valve leaflet; 310, free edge; 320, fixed edge; 400, balloon; 500, support column; 600, support member.

### DESCRIPTION OF EMBODIMENTS

Technical solutions of embodiments of the present disclosure will be clearly and completely described below in combination with the drawings. Obviously, the described embodiments are only part of, rather than all of the embodiments of the present disclosure. Based on the embodiments of this disclosure, all other embodiments obtained by those ordinary skilled in the art without any creative work shall fall within the protection scope of this disclosure.

In order to better describe and illustrate the embodiments of the present disclosure, reference may be made to one or more figures, but the additional details or examples used to describe the figures should not be construed as limiting the scope of the disclosure, the embodiments currently described, or the preferred embodiments of the present disclosure.

It should be noted that when a component is referred to as “connected” to another component, it may be directly connected to another component or there may be a middle component connected therebetween. When a component is considered to be “set on” another component, it may be directly set on another component or there may be a middle component arranged therebetween.

Unless defined otherwise, all technical and scientific terms used herein have the same meaning as commonly understood by one of ordinary skill in the art to which this disclosure belongs. The terms used in the specification of the present disclosure are only for the purpose of describing specific embodiments and are not intended to limit the present disclosure.

Disclosed is a polymer valve leaflet material made of polyurethane, wherein the polymer valve leaflet material has a tensile strength in a range of 35 MPa to 60 MPa, an elastic modulus in a range of 15 MPa to 40 MPa, a softness in a range of 20° to 50°, and a thickness in a range of 0.10 mm to 0.20 mm.

The polymer valve leaflet material is made of a polyurethane material with good biocompatibility and biostability, which are intrinsic properties of the material itself, and the method will not alter the inherent chemical properties of the material.

Compared to existing polyurethane materials, the valve leaflet material exhibits improved tensile strength through stretching, resulting in enhanced force tolerance and moderate increase in elastic modulus, which significantly reduces valve deformation during cardiac diastolic-systolic cycles. Meanwhile, the softness of the material is significantly increased due to the decrease in thickness, which allows for smoother opening and closing of the valve leaflet after fabrication and further improves hemodynamic performance.

The polymer valve leaflet material has permanent deformation in a range of 5% to 10%.

The polymer valve leaflet material demonstrates excellent creep resistance, with rapid recovery from tensile deformation and minimal permanent deformation. When used as the valve leaflet material, it prevents excessive elongation of the valve leaflets that could otherwise compromise valve performance.

The polymer valve leaflet material is anisotropic. Due to stretching, polyurethane exhibits a hard segment orientation parallel to the stretching direction, resulting in a significant increase in tear resistance perpendicular to the stretching direction. When preparing the valve leaflet, selectively aligning its free edge with the stretching direction substantially enhances the tear resistance of the valve leaflet material, greatly reduces the possibility of fatigue damage caused by free edge tearing, and thereby improves the fatigue resistance of the valve leaflet.

The valve leaflet material may also be used as a covering material for a prosthetic valve, or a material for positioning, occlusion, and closure on the prosthetic valve other than the stent.

A method for preparing a polymer valve leaflet material includes the following steps:
applying force to the polymer membrane to stretch the polymer membrane to a target dimension in a first direction, the target dimension being 120% to 300% of the original dimension; while restraining the deformation of the polymer membrane in a second direction, the second direction being perpendicular to the first direction; and continuously applying the force for at least 30 minutes after the polymer membrane reaches the target dimension.

The preparation method of the polymer valve leaflet material provided in this disclosure only involves stretching treatment after preparing the polyurethane membrane. Without altering the inherent excellent biocompatibility and biostability of the polymer membrane, the physical properties of the polymer valve leaflet material are improved through physical processing.

In the present disclosure, by stretching, the slippage of soft segments with high mobility is manifested in advance, and the orientation of hard segments under stress is induced. Additionally, micro-crystallization zones are formed in some soft segments due to the tensile force, and more hydrogen bonds are formed between the soft and hard segments in the stretching direction, which further restrains the mobility of the soft segments, and reduces the valve leaflet material's propensity for further creep, that is, improving its creep resistance. This mitigates the risk of poor valve closure and regurgitation caused by excessive elongation due to creep. Moreover, the fracture resistance of material will not be compromised by stretching, and even increases due to local crystallization of soft segments. With the decrease in total thickness, the tensile strength of the material is increased dramatically and the fatigue resistance of the valve is significantly improved. Meanwhile, the reduced thickness improves the softness of the valve, resulting in a smoother opening and closing of the valve leaflets and a significant increase in fluid performance.

The deformation of polymer materials generally proceeds through four successive stages. In the first stage, deformation is caused by the change of bond length and bond angle within the molecular chain, which is instantaneous and reversible, with a small deformation amount and a large elastic modulus. The second stage involves viscoelastic deformation, arising from conformational changes of the molecular chain. This deformation requires a certain relaxation time, exhibits large deformation, and has a low elastic modulus. Although still reversible, it is accompanied by a certain degree of viscous flow. The third stage involves viscous flow, a deformation induced by the relative sliding between the molecular chains, which will progress indefinitely over time. The fourth stage involves permanent deformation, where the relative slippage between the molecular chains is completed and irreversible.

The creep of polymer material primarily results from the relative movement between internal molecular chains. The creep resistance of polymer material depends mainly on the relative mobility of the internal molecular chains. In the present disclosure, the polymer membrane is made of polyurethane, a thermoplastic elastomer with a structure of alternating soft and hard segments. The deformation under the action of tension occurs through the rearrangement of the soft and hard segments. At the instant of applying stress (deformation of the first stage), changes in bond angles and bond lengths within the soft and hard segments lead to elastic deformation, which is reversible. Subsequently, the conformation of the soft segment molecular chain changes (deformation of the second stage). This conformation change is also reversible. Meanwhile, the hard segments are oriented (deformation of the third stage), and some soft segments with more mobility are not restrained by the hard segment to be more mobile, undergo slippage and deformation (deformation of the fourth stage). This deformation is irreversible and, once the stress is removed, becomes permanent.

The orientation of the hard segments in the polyurethane molecular chain and the trace micro-crystallization of some soft segments under tensile force improve the creep resistance and mechanical properties of the material. During stretching, the soft segments with strong mobility undergo sufficient slippage, which effectively dissipates the creep. In addition, due to the orientation along the stretching direction, more hydrogen bonds are formed between the soft and hard segments, which increases the difficulty of further sliding and enhances the creep resistance of polyurethane materials.

In this disclosure, the target dimension of the polymer membrane after stretching is controlled within 120% to 300% of the original dimension, to ensure complete orientation of the hard segments (i.e., achieving deformation of the third stage), which eliminates creep caused by the orientation of hard segments in molecular chains. Meanwhile, soft segments with more mobility undergo sufficient slippage, which effectively dissipates the creep. Moreover, further slippage of soft segments is restrained by hydrogen bonds between soft and hard segments, significantly increasing the resistance to relative molecular chain movement. Consequently, once the slippage of small-molecular chain segments has been effectively suppressed, the creep caused by the orientation of the stretched hard segments is eliminated. Coupled with hydrogen bonding, this approach prevents excessive elongation of the valve leaflet material and significantly enhances its creep resistance, avoiding further elongation of the valve leaflet after implantation.

When the polymer membrane is stretched to a target dimension less than 120% of the original dimension, the tensile strength increases only slightly. When the target dimension exceeds 300% of the original dimension, the tensile strength increases significantly, so it is necessary to significantly reduce the thickness of the polymer membrane to retain adequate softness, given that the elastic modulus is proportional to the cube of the thickness. In case the polymer membrane is excessively thin, even minor variations in thickness can exert a pronounced influence on the overall performance, thereby adversely affecting the performance of the valve leaflet.

Upon reaching the target dimension, the force is continuously applied to the polymer membrane for at least 30 minutes to allow the hard segments therein ample time to complete the orientation. During this period, the applied force is dynamically adjusted to maintain the target dimension without further stretching.

After stretching, the thickness of the polymer membrane decreases, and micro-crystallization zone forms between the soft segment molecular chains, which leads to a slight increase in the maximum breaking force and an improvement in tensile strength. Meanwhile, the reduction in thickness significantly enhances the softness of the polymer membrane. In addition, stretching induces molecular orientation within the polymer membrane, and most of the molecular chains tend to be parallel to the stretching direction, resulting in a significant improvement in tear resistance perpendicular to that direction. Moreover, since the easily movable macromolecular segments have already fully extended during stretching, the further deformation of the material is mostly elastic deformation, which is specifically manifested as a significant decrease in the permanent deformation of the polymer membrane after stretching. As such, the performance of the polymer membrane is improved by stretching.

Under the tensile force, the molecular chains of the polymer membrane tend to align with the force direction, and the shear resistance of the polymer membrane is greatly improved in the direction perpendicular to the stretching direction (i.e., the first direction).

The polymer membrane is made of polyurethane, whose polyurethane molecular chains includes soft segments and hard segments, which are interlaced to form physical cross-linking points. When subjected to external forces, the weak physical cross-linking points are disrupted, causing early manifestation of potential creep. Meanwhile, the molecular orientation induced by stretching and hydrogen bonding interactions collectively enhances the creep resistance of the polymer valve leaflet material during using.

In the present disclosure, the polyurethane material with good biocompatibility and biostability is utilized. Through stretching, the material achieves a substantial increase in tensile strength while maintaining the same level of softness, along with significant improvements in fatigue resistance. The elongated polyurethane exhibits markedly enhanced elasticity, a notable reduction in permanent deformation, and superior creep resistance. Additionally, the molecular chain orientation induced by stretching imparts anisotropic properties to the material, making it especially well-suited to the high shear resistance requirements of the free edge of the valve.

Compared with stereo molding, the valve leaflet preparation method provided by the present disclosure benefits from the substantially improved strength of polyurethane, allowing the valve to be prepared in the same manner as current bioprosthetic valves. This approach offers mature and stable processing, simple operation, and lower production costs, while achieving exceptional thickness uniformity of the valve. Stereo molding employs a stereoscopic leaflet design to substantially reduce internal stress during fatigue, compensating for its inherent strength limitations. However, the stereo molding involves expensive equipment, complex processes, and suffers from inconsistent thickness of the valve due to gravity effects, along with internal weak points. Moreover, it fails to address the creep issue inherent in polymer materials.

Compared with fabric reinforcement, the valve leaflet preparation method provided in the present disclosure eliminates concerns over fabric exposure caused by polyurethane abrasion and avoids the formation of free-edge notches.

The polymer membrane is elongated at a rate in a range of 100 mm/min to 500 mm/min.

The polymer membrane is elongated at a rate in a range of 100 mm/min to 200 mm/min.

The target dimension of the polymer membrane is 150% to 200% of the original dimension.

After the polymer membrane reaches the target dimension, the applied force is maintained for 30 minutes to 180 minutes.

The polymer membrane before being elongated has a thickness in a range of 0.15 mm to 0.40 mm.

The polymer membrane before being elongated has a thickness in a range of 0.15 mm to 0.30 mm.

The polymer membrane before being elongated has a tensile strength in a range of 20 MPa to 35 MPa, an elastic modulus in a range of 10 MPa to 30 MPa, a softness in a range of 10° to 20°, and a permanent deformation in a range of 10% to 40%.

The polymer membrane before being elongated is prepared by a casting molding process using polyurethane.

The polymer membrane is in a planar or curved shape during the stretching process.

During the stretching process, two opposite side edges of the polymer membrane are interconnected to form a circumferentially closed tubular structure.

The two opposite side edges are integrally connected or indirectly connected via a connecting member.

When the edges are indirectly connected, the polymer membrane spans at least half of the circumference of the tubular structure, with the remaining portion provided by the connecting member.

The polyurethane molecular chain includes hard and soft segments, wherein the soft segment content is in a range of 40% to 70%, and the rest is the hard segment. The soft segment is at least one selected from the following: polyether diol, polycarbonate diol, polyester diol, and polysiloxane diol, and the hard segment is isocyanate with an R value in a range of 1.0 to 1.1.

In the polyurethane molecular chain, the soft segment content is in a range of 50% to 65%.

The isocyanate is at least one selected from the following: TDI, HDI, MDI, NDI, PPDI, IPDI, and XDI.

The polyurethane molecular chain further includes a chain extender, and the chain extender is at least one selected from the following: ethylene glycol, butanediol, hexanediol, octanediol, and ethylenediamine.

The polymer membrane is planar and prepared by casting in a mold, wherein the mold has a flat bottom surface and side walls standing on the bottom surface and defining the boundaries of the polymer membrane.

A polyurethane solution with a concentration in a range of 3 wt. % to 40 wt. % is poured into the mold, and the solvent is evaporated to obtain the polymer membrane.

The polyurethane solution has a concentration in a range of 5 wt. % to 30 wt. %.

The solvent of the polyurethane solution is volatilized at 30° C. to 100° C. in a nitrogen atmosphere.

The solvent of the polyurethane solution is at least one selected from the following: DMAc, DMF, DMSO, toluene, and tetrahydrofuran.

The polymer membrane is tubular, and the method for preparing the tubular polymer membrane includes:

Step 1, applying a polyurethane solution onto the surface of the tubular mold and volatilizing the solvent to obtain a polyurethane membrane; and

Step 2, repeating step 1 three to six times to obtain a tubular polymer membrane with a predetermined thickness on the surface of the tubular mold.

In step 1, the polyurethane solution is applied to the surface of the tubular mold, which can be achieved by at least one of coating and dipping.

In step 1, the tubular mold is rotated continuously at a speed in a range of 1 r/min to 30 r/min while being soaked in the polyurethane solution to achieve uniform coverage of the polyurethane solution on the surface of the tubular mold.

In step 1, the solvent is volatilized under a nitrogen atmosphere and dried at 30° C. to 100° C.

After the preparation, the tubular polymer membrane is cut into several valve leaflets that are integrally connected. Those valve leaflets collectively constitute a one-piece valve, which is then sutured to a stent. To accommodate stents of different sizes, the tubular mold has a diameter ranging from 15 mm to 35 mm.

The tubular mold is soaked in water for 1 hour to 12 hours together with the tubular mold, and the tubular polymer membrane is peeled off from the surface of the tubular mold.

The tubular mold is a rotatable body, with its generatrix being straight or curved.

The rotation axis of the tubular mold is arranged horizontally.

The cross section of the tubular mold is not limited to being circular, but may also be configured with a smoothly extending contour such as elliptical.

The first direction and the second direction are coplanar and perpendicular, or the first direction and the second directions are arranged in three-dimensional space, where one of the first and second directions corresponds to the axial direction while the other of the first and second directions corresponds to a circumferential direction around that axis.

The deformation of the polymer membrane is W1 (deformation W1=value of dimensional change in the first direction/initial length) when it is elongated to the target dimension in the first direction, the deformation of the polymer membrane in the second direction is W2 (deformation W2=absolute value of dimensional change in the second direction/initial length), wherein W2/W1<30%. More preferably, W2/W1<10%. Even more preferably, W2/W1<5%.

The deformation of the polymer membrane in the second direction is restrained by applying a restraining force to two opposite sides of the polymer membrane in the second direction.

In terms of control implementation, the process can be controlled based on the deformation magnitude in the second direction. Optionally, when applying the restraining force, the two opposite sides of the polymer membrane in the second direction may be fixed, that is, maintaining zero deformation in the second direction. Alternatively, the variation of deformation in the second direction may be controlled according to a first trend, e.g., the deformation per unit time in the second direction. Of course, to accommodate variation in deformation, the magnitude of the restraining force is adjusted accordingly.

Furthermore, the control may be implemented based on the magnitude of the restraining force. Optionally, the magnitude of the restraining force may be constant or controlled according to a second trend.

For example, the second trend may be defined as the rate of change in restraining force per unit time. In case a constant restraining force is applied, the deformation in the second direction is passively adapted. In case the restraining force is actively adjusted, the deformation becomes actively controlled. Various approaches for restraining the deformation of the polymer membrane in the second direction may be employed in combination.

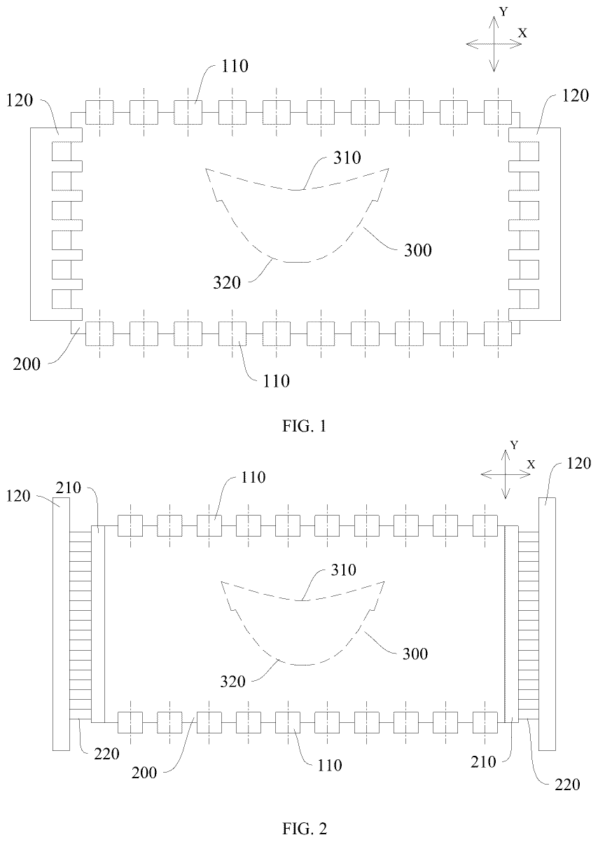

Referring to FIG. 1 and FIG. 2, for a planar polymer membrane, the polymer membrane is fixed in a second direction; and
a force is applied to the polymer membrane in a first direction, wherein the first direction is perpendicular to the second direction.

The polymer membrane is fixed in the second direction to maintain its original dimension as much as possible in the second direction. The force is applied in the first direction to elongate the polymer membrane and reduce its overall thickness.

When the force is applied to the polymer membrane in the first direction, the polymer membrane elongates in the first direction, and the means for fixing the polymer membrane in a second direction do not prevent this elongation but rather accommodates it.

Referring to FIG. 1 and FIG. 2, the angle between the first direction and the second direction is 90°. In FIG. 1, the X-direction corresponds to the first direction, and the Y-direction corresponds to the second direction. Taking FIG. 1 as illustrative, in this disclosure, the first direction refers to the direction along the X-axis, either leftward or rightward, and the second direction refers to the direction along the Y-axis, either upward or downward.

Referring to FIGS. 1 and 2, a first device is used to elongate the planar polymer membrane 200, and the first device includes:
a first clamp (providing the force that restrains deformation in the second direction) for fixing the polymer membrane 200 in the second direction; and
a second clamp 120 used for applying the force to the polymer membrane 200 in the first direction.

Optionally, the first clamp provides multiple force-applying sites for the same-side edge of the polymer membrane, and the distance between the force-applying sites along the first direction is adjustable.

The adjustment of distance may be either passively following or active. For active distance adjustment, it is preferably to synchronize with the deformation speed of the polymer membrane in the first direction, that is, the distance change of the first clamp is consistent with the deformation speed of the polymer membrane in the first direction, ensuring that the distance adjustment of the first clamp neither impedes nor promotes the deformation process of the polymer membrane in the first direction.

The first clamp and the second clamp engage with the corresponding force-applying sites in the polymer membrane. For some force-applying sites, for example, the corners of the rectangular membrane, the first clamp and the second clamp may simultaneously engage with the force-applying site. In this case, it is understood that one of the first clamp and the second clamp serves dual functions for the other.

The first fixture is a press roller 110. There are two sets of press rollers 110, each corresponding to one of the two opposite edges of the polymer membrane 200, with the axial direction of each press roller 110 parallel to the second direction.

Each set of press rollers 110 includes at least two press rollers 110. The contact areas where the press rollers 110 interact with the polymer membrane 200 defines contact lines, and the distance between the contact lines of two adjacent press rollers 110 is in a range of 5 mm to 20 mm.

Each press roller 110 includes a fixed shaft and a rotating roller rotatably mounted around the fixed shaft.

There are two pairs of second clamps 120, each corresponding to one of the two opposite edges of the polymer membrane 200, and the two pairs of second clamps 120 clamp the polymer membrane 200 and move in opposite directions to elongate the polymer membrane 200 in the first direction.

Each second clamp 120 includes multiple clamping parts arranged at intervals, and the interval between two adjacent clamping parts ranges from 5 mm to 20 mm.

The two pairs of second clamps 120 move in opposite directions at the identical speeds. The combined movement speeds of the two pairs of second clamps 120 equal the elongation rate of the polymer membrane 200. For example, if the elongation rate of the polymer membrane 200 is 100 mm/min, the movement speed of each second clamp 120 is 50 mm/min.

Alternatively, in case one of the two pairs of second clamps remains stationary while the other pair moves, the movement speed of the moving second clamp corresponds directly to the elongation rate of the polymer membrane.

As shown in FIG. 2, two opposite edges of the polymer membrane 200 have fabric-reinforced borders 210, and the fabric-reinforced borders 210 are connected with traction lines 220 for engaging with the second clamp 120.

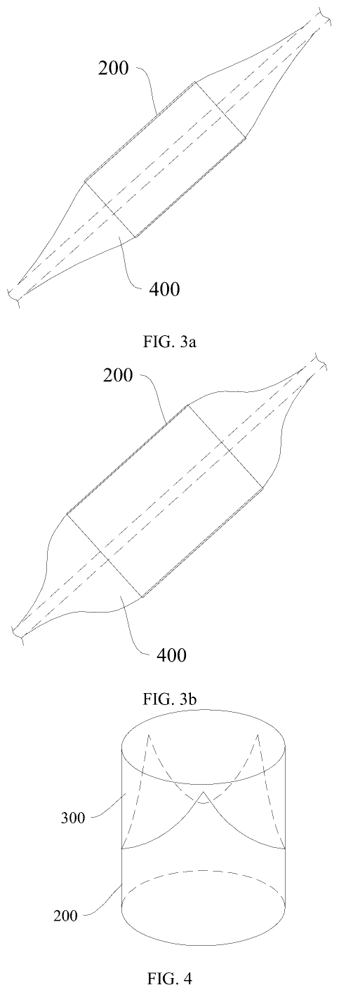

Referring to FIGS. 3a and 3b, a second device is used to elongate the tubular polymer membrane, wherein the second device includes:
a radially expandable balloon 400, having a tubular segment around which the tubular polymer membrane is mounted; and
an infusion device provided for delivering fluid into the balloon 400.

The first direction may be understood as the circumferential direction of the tubular segment, and the second direction may be understood as the axial direction of the tubular segment.

The two axial sides of the tubular polymer membrane may be fixed to the outer periphery of the tubular segment, for example, by means of adhesion.

As shown in FIG. 3a, the tubular polymer membrane 200 is mounted around the tubular segment of the balloon 400. The infusion device delivers fluid (which may be gas or liquid) into the balloon 400, causing the balloon 400 expand radially. As a result, the polymer membrane mounted around the tubular section of the balloon 400 also undergoes deformation (i.e., transitioning from the state in FIG. 3a to that in FIG. 3b), achieving elongation along the circumferential direction of the cylindrical segment.

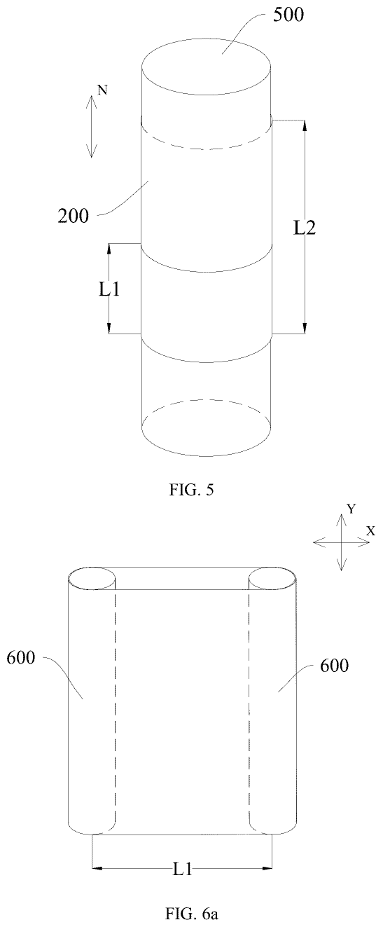

As shown in FIG. 5, a third device is used to elongate the tubular polymer membrane, and the third device includes:
a support column 500, configured to mount the tubular polymer membrane thereon;
two clamping members, each clamping one axial end of the polymer membrane to elongate it in a first direction (i.e., N-direction in the figure); and
at least one movable ring slidably mounted around the support column, with at least one clamping member disposed on the movable ring.

The first direction may be understood as the axial direction of the support column, and the second direction may be understood as the circumferential direction of the support column.

The cross section of the support column is not strictly limited and may adopt a configuration with a smooth outer contour such as a circle or ellipse. The support column may be solid or hollow, as long as it provides sufficient structural strength.

The movable ring slides along the axial direction of the support column to elongate the tubular polymer membrane in the first direction. The support column restrains the radial contraction of the polymer membrane, while the dimension of the polymer membrane in the circumferential direction remains unchanged. As shown in FIG. 5, the axial length of the polymer membrane is elongated from L1 to L2.

Two movable rings are mounted around the support column, with each clamping member disposed on the corresponding movable ring.

A movable ring is mounted around the support column, with one clamping member fixed to the support column and the other fixed to the movable ring.

The support column is provided with a guide device for defining the path of the movable ring as axial along the support column.

Two axial ends of the tubular polymer membrane are provided with fabric-reinforced borders, respectively, and the fabric-reinforced borders are connected with traction lines for cooperating with the clamping member.

The cross section of the support column may be circular or configured with a smoothly extending contour, such as elliptical.

Optionally, a fourth device is used to elongate the tubular polymer membrane, and the fourth device includes:
multiple support members movable toward and away from each other, each support member being acting on the inner surface of the tubular polymer membrane; and
a drive mechanism for driving at least two support members to move away from each other.

When the fourth device is used, the tubular polymer membrane is mounted around the outer periphery of multiple support members; that is, the multiple support members are all located inside the tubular polymer membrane, and the driving mechanism is used to drive at least two support members away from each other to elongate the polymer membrane.

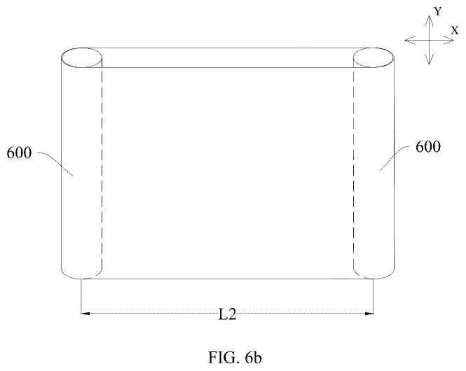

Taking two support members as an example, as shown in FIG. 6a and FIG. 6b, a tubular polymer membrane is mounted around the outer peripheries of two support members 600, and a driving mechanism (not shown in the figure) is used to drive the two support members 600 to move away from each other. In the first direction (i.e., the X-direction in the figure), the dimension of the polymer membrane changes from L1 to L2. In the second direction, i.e., the Y-direction, the dimension of the polymer membrane remains fixed.

A method for preparing a polymer valve leaflet includes:
soaking the polymer valve leaflet material in water for at least 4 hours and cutting it to obtain the polymer valve leaflet.

When preparing the valve leaflet, the extending direction of the free edge is aligned with the stretching direction. Under applied force, the hard segment of the polyurethane material will be oriented and crystallized along the stretching direction. This significantly enhances the tear resistance in the vertical direction and greatly reduces the fatigue failure of the valve leaflet caused by tearing of the free edge.

The polymer valve leaflet material is soaked in water at 30° C. to 40° C.

The polymer valve leaflet material was soaked in water at 37° C.

When preparing the polymer valve leaflet, the polymer valve leaflet material is soaked in water at 37° C. to simulate physiological conditions. This allows complete stress relaxation in the polymer valve leaflet material, preventing deformation after implantation.

As shown in FIGS. 1 and 2, the valve leaflet 300 includes a fixed edge 320 fixedly connected to the stent and a free edge 310 that cooperates with other valve leaflets to control the blood flow channel. The polymer membrane is elongated in a first direction, and the first direction is parallel to the extending direction of the free edge 310 of the valve leaflet.

Referring to FIGS. 1 and 2, the outline of the valve leaflet 300 is indicated by a dotted line. The valve leaflet 300 is cut from the valve leaflet material along the dotted line in the middle portion of the valve leaflet material to obtain the valve leaflet.

Optionally, the most uniform portion in the middle of the elongated polymer membrane is selected for cutting.

Optionally, when cutting the valve leaflets, it should be ensured that the free edge aligns with the first direction, so that the direction of force applied to the valve leaflets is parallel to the stretching direction of the polymer membrane.

The polymer membrane is elongated in the first direction, such that the molecular chains are oriented in the first direction. The first direction is parallel to the extending direction of the free edge of the valve leaflet; that is, the free edge runs parallel to the extension direction of the molecular chains. When the free edge is subjected to external force, the molecular chain itself is highly resistant to rupture, so the free edge exhibits better tear resistance and will not develop a central notch during leaflet fatigue.

The polymer valve leaflet material has a thickness in a range of 0.10 to 0.20 mm.

The polymer valve leaflet material has a tensile strength in a range of 35 MPa to 60 MPa, an elastic modulus in a range of 15 MPa to 40 MPa, a softness in a range of 20 to 50°, and a permanent deformation in a range of 5% to 10%.

A polymer valve leaflet is prepared by the aforementioned polymer valve leaflet preparation method.

The polymer valve leaflet material has a tensile strength in a range of 35 MPa to 60 MPa, an elastic modulus in a range of 15 MPa to 40 MPa, a softness in a range of 20 to 50°, and a permanent deformation in a range of 5% to 10%.

A polymer valve includes:
a stent, with a blood flow channel defined therein; and
one or more valve leaflets, wherein the valve leaflets are made of the polymer valve leaflet material.

The polymer valve leaflet material has a tensile strength in a range of 35 MPa to 60 MPa, an elastic modulus in a range of 15 MPa to 40 MPa, a softness in a range of 20 to 50°, and a permanent deformation in a range of 5% to 10%.

The valve includes multiple valve leaflets, which may be prepared individually and subsequently joined by suturing. The valve leaflets may also be prepared at one time. For example, a tubular polymer membrane, after stretching, forms a membrane structure as shown in FIG. 4. This tubular membrane can either be cut directly into an integral multi-leaflet configuration, or sectioned into discrete individual valve leaflets that are subsequently joined via suturing. The valve leaflets cut from the tubular polymer membrane have a three-dimensional configuration, which can better adapt to the stent during actual use and have better mechanical properties.

A method for preparing a polymer valve leaflet material includes the following steps:
(1) dissolving the polyurethane in at least one of the following: DMAc, DMF, DMSO, toluene and tetrahydrofuran;
(2) pouring 30 mL to 400 mL of polyurethane solution with a concentration in a range of 3 wt. % to 40 wt. % into a mold, which is a square box with a bottom area of 200 mm*200 mm;
(3) baking the mold at 60° C. to 80° C. under a dry nitrogen atmosphere for 6 to 24 hours, to form a polyurethane membrane (i.e., polymer membrane) with a thickness in a range of 0.15 mm to 0.3 mm; and
(4) elongating the polyurethane membrane by a stretching device (first device). As shown in FIG. 1, the polyurethane membrane is rectangular, with two opposite edges clamped by rollers 110, respectively. The roller 110 restrains the movement of the polyurethane membrane in the second direction (i.e., the Y-direction). The other two opposite edges of the polyurethane membrane are clamped by corresponding clamps 120, which move in opposite directions to stretch the polyurethane membrane in the first direction. The rollers 110 are rotatably displaceable relative to the polyurethane membrane to accommodate its deformation in the first direction. The elongation rate ranges from 50 mm/min to 500 mm/min, with both clamps on opposite edges advancing at the same rate. When the membrane is elongated to 120% to 300% of the original length, the clamps are secured and held for 30 minutes to 180 minutes.
(5) When cutting the valve leaflets, it is ensured that the free edge extends in line with the first direction.

A method for preparing a polymer valve leaflet material includes the following steps:
(1) dissolving the polyurethane in at least one of the following: DMAc, DMF, DMSO, toluene and tetrahydrofuran;
(2) placing a frame-shaped (rectangular frame) fabric with a frame width of 1 mm to 5 mm in a mold. The mold is a square box with a bottom area of 200 mm*200 mm; and pouring 30 mL to 400 mL of polyurethane solution with a concentration of 3 wt. % to 40 wt. % into the mold.
(3) baking the mold at 60° C. to 80° C. under a dry nitrogen atmosphere for 6 hours to 24 hours, to form a polyurethane membrane (i.e., polymer membrane) with a thickness in a range of 0.15 mm to 0.3 mm; and
(4) trimming the polyurethane membrane to remove the polyurethane lying outside the frame-shaped fabric. The resulting polyurethane membrane is rectangular, and the fabric at two opposite edges thereof is cut to accommodate the subsequent stretching process of the polyurethane membrane. The fabric at the remaining two opposite edges serves as the fabric-reinforced border 210.

The fabric-reinforced borders 210 of the two opposite uncut edges of the polyurethane membrane are connected with stitches (i.e., traction lines 220), and the stitches are connected to the clamps. The stretching process is illustrated in FIG. 2. The two opposite edges of the polyurethane membrane without fabric are clamped by the rollers 110, respectively. The roller 110 restrains the movement of the polyurethane membrane in the second direction (i.e., the Y-direction). Stitches connected to the other two opposite edges of the polyurethane membrane are clamped by corresponding clamps 120, which move in opposite directions to stretch the polyurethane membrane in the first direction. The rollers 110 are rotatably displaceable relative to the polyurethane membrane to accommodate its deformation in the first direction. The elongation rate is in a range of 50 mm/min to 500 mm/min, with both clamps on opposite edges advancing at the same rate. When the membrane is elongated to 120% to 300% of the original length, the clamps are secured and held for 30 minutes to 180 minutes.
(5) When cutting the valve leaflets, it is ensured that the free edge extends in line with the first direction.

A method for preparing a polymer valve leaflet material includes the following steps:
(1) dissolving the polyurethane in at least one of the following: DMAc, DMF, DMSO, toluene and tetrahydrofuran;
(2) soaking a tubular mold (15 mm to 35 mm in diameter) rotating at a speed in a range of 1 r/min to 30 r/min into the polyurethane solution;
(3) drying the mold at 60° C. to 80° C. under a dry nitrogen atmosphere;
(4) repeating steps (2) to (3) generally for 3 to 6 cycles based on the deposited thickness of the polyurethane on the tubular mold;
(5) soaking the tubular mold in pure water for 1 to 12 hours to peel off the polyurethane membrane from the surface of the tubular mold; and
(6) mounting the polyurethane membrane around the balloon, inflating the balloon until its diameter increases to 120% to 300% of the mold diameter (i.e., reaching the state shown in FIG. 3b), as shown in FIG. 3a; and maintaining the expansion for 30 min to 180 min.
(7) Referring to FIG. 4, the polyurethane material is cut into a leaflet shape.

A method for preparing a polymer valve leaflet material includes the following steps:
(1) dissolving the polyurethane in at least one of the following: DMAc, DMF, DMSO, toluene and tetrahydrofuran;
(2) soaking a tubular mold (15 mm to35 mm in diameter) rotating at a speed of 1 r/min to 30 r/min into the polyurethane solution;
(3) drying the mold at 60° C. to 80° C. under a dry nitrogen atmosphere;
(4) repeating steps (2) to (3) generally for 3 to 6 cycles based on the deposited thickness of the polyurethane on the tubular mold;
(5) soaking the tubular mold in pure water for 1 hour to 12 hours to peel off the polyurethane membrane from the surface of the tubular mold;
(6) stretching the polyurethane membrane by the third device along the axial direction until its axial length reaches 120% to 300% of the pre-stretched length, as shown in FIG. 6, and maintaining the state for 30 minutes to 180 minutes; and
(7) cutting the tubular polymer membrane along the axial direction and spreading it flat.
(8) When cutting the valve leaflets, it is ensured that the free edge extends in line with the first direction.

### 258. Example 1

A method for preparing a polymer valve leaflet material includes the following steps:
(1) Synthesizing PDMS-PU (i.e., polyurethane), wherein the polyurethane has a block composition of 32.23% MDI, 11% PHMO, 44% PDMS, 10.5% BHTD, and 2.27% EDA. The tensile strength of PDMS-PU is 28 MPa, and the elastic modulus is 15 MPa;
(2) Dissolving PDMS-PU in DMAc to configure a 10 wt. % polyurethane solution;
(3) Pouring 100 mL of polyurethane solution into a mold (200 mm*200 mm metal box);
(4) Baking the mold at 80° C. under a dry nitrogen atmosphere for 12 hours to form a polyurethane membrane (i.e., polymer membrane) with a thickness of 0.23 mm; and
(5) Stretching the polyurethane membrane. The stretching method is shown in FIG. 1. The polyurethane membrane is elongated by the clamps in the left-right direction at a rate of 100 mm/min. When the membrane is elongated to 200% of its original length, the clamp is secured and maintained for 120 min.
(6) When cutting the valve leaflets, it is ensured that the free edge extends in line with the first direction.

### Example 2

A method for preparing a polymer valve leaflet material includes the following steps:
(1) Synthesizing PDMS-PU (i.e., polyurethane), wherein the polyurethane has a block composition of 32.23% MDI, 11% PHMO, 44% PDMS, 10.5% BHTD, and 2.27% EDA. The tensile strength of PDMS-PU is 28 MPa, and the elastic modulus is 15 MPa;
(2) Dissolving PDMS-PU in DMAc to configure a 10 wt. % polyurethane solution;
(3) Placing a fame-shaped (rectangular frame) fabric with a frame width of 5 mm in a mold, wherein the mold is a square box with a bottom area of 200 mm*200 mm; and pouring 100 mL of polyurethane solution into the mold;
(4) Baking the mold at 80° C. under a dry nitrogen atmosphere for 12 hours to form a polyurethane membrane (i.e., polymer membrane) with a thickness of 0.25 mm; and
(5) Trimming the polyurethane membrane to remove the polyurethane lying outside the frame-shaped fabric. The polyurethane membrane is rectangular, and the fabric at two opposite edges thereof is cut to accommodate the subsequent stretching process of the polyurethane membrane. As shown in FIG. 2, the polyurethane membrane is elongated by the clamps in the right-left direction at an elongation rate of 100 mm/min. When the membrane is elongated to 200% of its original length, the clamp is secured and maintained for 120 minutes.
(6) When cutting the valve leaflet, it is ensured that the free edge extends in line with the first direction.

### Example 3

A method for preparing a polymer valve leaflet material includes the following steps:
(1) Synthesizing PDMS-PU (i.e., polyurethane), wherein the polyurethane has a block composition of 32.23% MDI, 11% PHMO, 44% PDMS, 10.5% BHTD, and 2.27% EDA. The tensile strength of the polyurethane is 28 MPa and the elastic modulus is 15 MPa;
(2) Dissolving polyurethane in DMAc to configure a 10 wt. % polyurethane solution;
(3) Soaking a tubular mold (diameter 18 mm) rotating at a speed of 5 r/min into the polyurethane solution;
(4) Drying the mold at 80° C. in a dry nitrogen atmosphere;
(5) Repeating steps 2 to 3 three times;
(6) Soaking the tubular mold in pure water for 6 hours to peel off the polyurethane membrane from the surface of the tubular mold;
(7) Mounting the polyurethane membrane around the balloon and inflating the balloon until its diameter increases to 200% of the mold diameter, and maintaining the expansion for 120 min, as shown in FIGS. 3a and 3b; and
(8) Cutting the polyurethane valve leaflet concerning FIG. 4.

### Example 4

A method for preparing a polymer valve leaflet material includes the following steps:
(1) Synthesizing PDMS-PU (i.e., polyurethane), wherein the polyurethane has a block composition of 32.23% MDI, 11% PHMO, 44% PDMS, 10.5% BHTD, and 2.27% EDA. The tensile strength of the polyurethane is 28 MPa and the elastic modulus is 15 MPa;
(2) Dissolving polyurethane in DMAc to configure a 10 wt. % polyurethane solution;
(3) Soaking a tubular mold (18 mm in diameter) rotating at a speed of 5 r/min into the polyurethane solution;
(4) Drying the mold at 80° C. under a dry nitrogen atmosphere;
(5) Repeating steps (2) to (3) three times;
(6) Soaking the tubular mold in pure water for 6 hours to peel off the polyurethane from the surface of the tubular mold;
(7) Referring to FIG. 5, stretching the tubular polymer membrane axially by a third device until its axial length reaches 120% to 300% of the pre-stretched length, and maintaining the state for 30 minutes to 180 minutes; and
(8) Cutting the tubular polymer membrane along the axial direction and spreading it flat.
(9) When cutting the valve leaflets, it is ensured that the free edge extends in line with the first direction.

### Control Example 1

A method for preparing a polymer valve leaflet material includes the following steps:
(1) Synthesizing PDMS-PU. The polyurethane has a block composition of 32.23% MDI, 11% PHMO, 44% PDMS, 10.5% BHTD, and 2.27% EDA. The tensile strength of polyurethane is 28 MPa and the elastic modulus is 15 MPa.
(2) Dissolving polyurethane in DMAc to configure a 10 wt. % polyurethane solution;
(3) Pouring 100 mL of polyurethane solution into a mold (200 mm*200 mm metal box); and
(4) Baking the mold at 80° C. under a dry nitrogen atmosphere for 12 h to form a polyurethane membrane with a thickness of 0.23 mm.

### Performance Characterization

Characterization methods:

Thickness: Measured with a thickness gauge with an accuracy of 0.01 mm. The thickness of each sample was measured 5 times and the average value was taken.

Tensile strength: Tested according to ASTM D412-16 standard. Test specimens were cut into Type 2 dumbbell shapes.

Shear strength: Tested according to ATSM D624-00 standard. Test specimens were cut into Die C shapes.

Softness: the valve leaflet material was cut into 10 mm×50 mm strips. Each strip was placed symmetrically on a test platform. The position was adjusted so that the strip hung down symmetrically. A straight line is drawn from the center point to one end of the strip, and the angle between this line and the horizontal was measured.

Permanent deformation: The valve leaflet material was cut into the type 2 dumbbell shapes according to ASTM D412-16 standard. Two parallel lines were marked on the narrow section of the dumbbell-shaped membrane, with a distance between the marking lines of 30 mm. The crosshead speed was set to 500 mm/min. The stretching was stopped when the distance reached 60 mm, and the specimen was removed. After allowing the specimen to rest for 10 minutes, the distance 1 between the marking lines was measured. The permanent deformation was calculated as (1−30)/30.

EOA (Effective Opening Area): Tested according to ISO 5840-3:2021 standard.

Fatigue resistance: Tested in accordance with ISO 5840-1:2021.

The performance characterization results of Example 1, Example 3 and Control Example 1 are shown in Table 1.

TABLE 1

| | | | | | EOA | | |
|---|---|---|---|---|---|---|---|
| | | | Longitudinal | | (Effective | | |
| Serial | | Tensile | Tear | | Opening | Permanent | Fatigue Resistance |
| No. | Thickness | Strength | Resistance | Softness | Area) | Deformation | Performance Test |
| Example 1 | 0.15 mm | 45 MPa | 80 N/mm | 30° | 2.2 | 6.5% | No failure after |
| | | | | | | | 600 million cycles |
| Example 3 | 0.15 mm | 46 MPa | 82 N/mm | 31° | 2.3 | 6.7% | No failure after |
| | | | | | | | 600 million cycles |
| Control | 0.23 mm | 28 MPa | 50 N/mm | 15° | 1.6 | 13.6% | Notch appeared at |
| Example 1 | | | | | | | free edge after 10 |
| | | | | | | | million cycles; |
| | | | | | | | Multiple sites |
| | | | | | | | fractured after 300 |
| | | | | | | | million cycles |

As shown in Table 1, after the stretching treatment, the polymer membrane exhibited significantly increased tensile strength and longitudinal tear resistance, along with improved softness (EOA is generally related to softness, the larger the EOA, the softer the material), and the fatigue resistance performance test was excellent.

The technical features of the above embodiments can be arbitrarily combined, and not all possible combinations of the technical features of the above embodiments have been described for the sake of brevity of description. However, as long as there is no contradiction in the combination of these technical characteristics, such combination should be regarded as falling into the scope of this specification.

The above-described embodiments only illustrate several embodiments of the present disclosure, and the descriptions thereof are specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure.

1. A method for preparing a polymer valve leaflet material, comprising the following steps:
applying a force to a polymer membrane to elongate the polymer membrane to a target dimension in a first direction;
while restraining deformation of the polymer membrane in a second direction perpendicular to the first direction, the target dimension being 120% to 300% of an original dimension of the polymer membrane; and
continuously applying the force to the polymer membrane for at least 30 minutes once the polymer membrane reaches the target dimension.

2. The method for preparing a polymer valve leaflet material according to claim 1, wherein the polymer membrane is elongated at a rate in a range of 100 mm/min to 500 mm/min.

3. The method for preparing a polymer valve leaflet material according to claim 1, wherein the polymer membrane before being elongated has a thickness in a range of 0.15 mm to 0.40 mm.

4. The method for preparing a polymer valve leaflet material according to claim 1, wherein the polymer membrane before being elongated has a tensile strength in a range of 20 MPa to 35 MPa, an elastic modulus in a range of 10 MPa to 30 MPa, a softness in a range of 10° to 20°, and a permanent deformation in a range of 10% to 40%.

5. The method for preparing a polymer valve leaflet material according to claim 1, wherein the polymer membrane before being elongated is made of polyurethane and formed by a casting molding process, wherein polyurethane molecular chains comprise hard segments and soft segments, and a content of the soft segments is in a range of 40% to 70%, wherein the target dimension of the polymer membrane is 150% to 200% of the original dimension, and the force is continuously applied for 30 minutes to 180 minutes once the polymer membrane reaches the target dimension.

6. The method for preparing a polymer valve leaflet material according to claim 1, wherein the polymer membrane is in a planar or curved shape before being elongated.

7. The method for preparing a polymer valve leaflet material according to claim 1, wherein during elongating, two opposite side edges of the polymer membrane are interconnected to form a tubular structure which is circumferentially closed; and
wherein the two opposite side edges are integrally connected; or the two opposite side edges are indirectly connected via a connecting member, in which the polymer membrane spans at least half of a circumference of the tubular structure, with a remaining portion of the circumference being constituted by the connecting member.

8. The method for preparing a polymer valve leaflet material according to claim 1, wherein first and second directions are coplanar and perpendicular; or the first and second directions are arranged in a three-dimensional space, with one of the first and second directions being corresponding to an axial direction while an other of the first and second directions being corresponding to a circumferential direction around the axial direction; and
wherein the deformation of the polymer membrane in the second direction is restrained by applying a restraining force to two opposite sides of the polymer membrane in the second direction.

9. The method for preparing a polymer valve leaflet material according to claim 1, wherein the deformation of the polymer membrane is W1 when the polymer membrane is elongated to the target dimension in the first direction, the deformation of the polymer membrane in the second direction is W2, and W2/W1<30%.

10. The method for preparing a polymer valve leaflet material according to claim 6, wherein the polymer membrane is a planar membrane or a curved membrane during elongating, a first device is used to elongate the planar polymer membrane, and the first device comprises:
a first clamp for fixing the polymer membrane in the second direction; and
one or more second clamps used for applying the force to the polymer membrane;
wherein the first clamp provides a plurality of force-applying sites for a same-side edge of the polymer membrane, and distance between the plurality of force-applying sites along the first direction is adjustable.

11. The method for preparing a polymer valve leaflet material according to claim 10, wherein the first clamp comprises two sets of press rollers, each corresponding to one of two opposite edges of the polymer membrane, wherein each of the two sets of press rollers comprises at least two press rollers, with an axial direction of each press roller parallel to the second direction, contact areas where the press rollers interact with the polymer membrane define contact lines, and distance between contact lines of two adjacent press rollers is in a range of 5 mm to 20 mm, wherein the each press roller comprises a fixed shaft and a rotating roller rotatably mounted on the fixed shaft.

12. The method for preparing a polymer valve leaflet material according to claim 10, wherein the one or more second clamps comprise two pairs of second clamps, each corresponding to one of two opposite edges of the polymer membrane, and the two pairs of second clamps clamp the polymer membrane and move in opposite directions to elongate the polymer membrane in the first direction, wherein each of the two pairs of second clamps comprises a plurality of clamping parts arranged at intervals, and distance between two adjacent clamping parts ranges from 5 mm to 20 mm.

13. The method for preparing a polymer valve leaflet material according to claim 6, wherein a second device is used to elongate a tubular polymer membrane, and the second device comprises:
a radially expandable balloon, having a tubular segment around which the tubular polymer membrane is mounted; and
an infusion device for delivering fluid into the balloon;
wherein two axial sides of the tubular polymer membrane are fixable to an outer periphery of the tubular segment.

14. The method for preparing a polymer valve leaflet material according to claim 6, wherein a third device is used to elongate a tubular polymer membrane, and the third device comprises:
a support column, configured to mount the tubular polymer membrane thereon;
two clamping members, each clamping one axial end of the polymer membrane to elongate the polymer membrane in the first direction; and
at least one movable ring, slidably mounted around the support column, and at least one of the clamping members is disposed on the at least one movable ring.

15. The method for preparing a polymer valve leaflet material according to claim 14, wherein two movable rings are mounted around the support column, with two clamping members respectively disposed on corresponding movable rings, or a movable ring is mounted around the support column, with one clamping member fixedly disposed on the support column and an other clamping member fixedly disposed on the movable ring; and
wherein the support column is further provided with a guide device for defining a path of the movable ring as axial along the support column.

16. The method for preparing a polymer valve leaflet material according to claim 6, wherein a fourth device is used to elongate a tubular polymer membrane, and the fourth device comprises:
a plurality of support members movable toward and away from each other, each support member being acting on an inner surface of the tubular polymer membrane; and
a drive mechanism for driving at least two support members to move away from each other.

17. The method for preparing a polymer valve leaflet material according to claim 1, further comprising soaking prepared polymer valve leaflet material in water at 30° C. to 40° C., and cutting soaked polymer valve leaflet material to obtain a polymer valve leaflet, wherein the polymer valve leaflet material has a thickness in a range of 0.10 mm to 0.20 mm.

18. A polymer valve leaflet material prepared by the method for preparing the polymer valve leaflet material according to claim 1.

19. The polymer valve leaflet material according to claim 18, which is made of polyurethane, and the polymer valve leaflet material has a tensile strength in a range of 35 MPa to 60 MPa, an elastic modulus in a range of 15 MPa to 40 MPa, a softness in a range of 20° to 50°, a permanent deformation in a range of 5% to 10%, and a thickness in a range of 0.10 mm to 0.20 mm.

20. A polymer valve, comprising:
a stent, defining a blood flow channel therein; and
one or more valve leaflets prepared by the method for preparing a polymer valve leaflet material according to claim 1.
